(19) United States
(12) Patent Application Publication
(10) Pub. No.: US 2022/0346884 A1
(43) Pub. Date: Nov. 3, 2022

(54) INTRAOPERATIVE IMAGE-GUIDED TOOLS FOR OPHTHALMIC SURGERY

(71) Applicant: MICROSURGICAL GUIDANCE SOLUTIONS, LLC, Chicago, IL (US)

(72) Inventors: YANNEK I. LEIDERMAN, RIVER FOREST, IL (US); ROGERIO GARCIA NESPOLO, WARRENVILLE, IL (US)

(73) Assignee: MICROSURGICAL GUIDANCE SOLUTIONS, LLC, Chicago, IL (US)

(21) Appl. No.: 17/735,079

(22) Filed: May 2, 2022

Related U.S. Application Data
(60) Provisional application No. 63/183,424, filed on May 3, 2021.

(51) Int. Cl. A61B 34/20; A61F 9/007; A61B 34/37; G02B 21/00

(52) U.S. Cl. CPC A61B 34/20; A61F 9/007; A61B 34/37; G02B 21/0012; A61B 2034/254

(57) ABSTRACT

An image-guided tool and method for ophthalmic surgical procedures is disclosed comprising a processor, a display, an imaging system, and a memory communicatively coupled to the processor. The memory stores instructions executable by the processor and includes an artificial intelligence (AI) model. The processor is arranged to receive from the imaging system visual images in real-time of a surgical field during the ophthalmic surgical procedure and using the AI model to extract regions of interest in the surgical field. Upon selection of a region of interest by the AI model, the AI model develops operating image features based on the surgical instruments used in the region of interest and the phase of the surgical procedure being performed. Augmented visual images are then constructed that include the real-time visual image and the image features and surgical phase information. The augmented image is displayed on the display.

### CROSS-REFERENCE TO RELATED APPLICATION AND PRIORITY CLAIM

This application claims priority under 35 U.S.C. § 119(e) to U.S. Provisional Patent Application No. 63/183,424 filed on May 3, 2021. This provisional application is hereby incorporated by reference in its entirety.

### TECHNICAL FIELD

This disclosure is generally directed to assisted systems for ophthalmic surgical procedures. More specifically, this disclosure is directed to image-guided tool for cataract surgical procedures using an artificial intelligence (AI) model.

### BACKGROUND

Cataract extraction with lens implantation and vitrectomy procedures are among the most frequently performed ophthalmic surgeries in the United States and abroad. Although these procedures are generally considered safe and effective, surgical complications remain a cause of postoperative visual loss, including but not limited to retinal detachment, macular edema, intraocular bleeding, glaucoma, and permanent loss of vision.

All surgical procedures include inherent risk to the patient, the mitigation of which is an area of constant research and development. One advance in the field of medical practice has been to move to minimally invasive surgeries that do not require large incisions, and generally result in faster recovery, less pain, and less risk of complications. Many minimally invasive surgeries involve the insertion of one or more surgical instruments through one or more small incisions. Such surgeries generally rely on cameras, microscopes, or other imaging techniques (X-ray, ultrasound, etc.) in order for a surgeon performing the surgery to visualize the surgical field. However, one difficulty encountered during such procedures is that it can be difficult to interpret with accuracy visualization of the surgical field provided by medical imaging modalities to the surgeon.

In intraocular (i.e., within the eye) surgery, surgical optical visualization systems are used from outside of the eye to view the surgical field. Such visualization systems can include surgical microscopes (SM) or an optical coherence tomography (OCT) imaging system. OCT is an imaging technique that uses reflections from within imaged tissue to provide cross-sectional images. Unfortunately, OCT systems lack needed precision regarding the precise location of the surgical instruments and tools and are adversely affected by imaging artifacts such as for example, shadows induced by the material properties of the surgical instruments. Additionally, delays in the visual output, due to computational complexities, fails to visualize the surgical field in real-time.

Visual images from a surgical microscope (SM) can be difficult for a surgeon to interpret with accuracy and in real-time due to the relationship between the surgical instruments and the anatomical structures and tissues in proximity to the surgical instruments particularly when the surgical field is exceedingly small, such as when operating on the eye.

The present disclosure therefore discloses an image-guided tool and method that uses artificial intelligence (AI) model to post-process visual images acquired in real-time by an imaging system to provide visual and other feedback to the surgeon for the guidance and positioning of the surgical instruments as well as to provide warning of eye movement and information on tissue segmentation.

### SUMMARY

This disclosure relates to image-guided tools for cataract surgical procedures using deep learning and computer vision in ophthalmic surgical procedures using an artificial intelligence (AI) model.

In a first embodiment, an image-guided tool for surgical procedures is disclosed comprising, a processor, a display device, an imaging system, and a memory communicatively coupled to the processor. The memory stores instructions executable by the processor and includes an artificial intelligence (AI) model. The processor is arranged to receive from the imaging system visual images in real-time of a surgical field during the surgical procedure and using the AI extract regions of interest in the surgical field. Upon selection of a region of interest computed by the AI, the AI develops operating image features based on surgical instrument used in the region of interest and the phase of the surgical procedure being performed. Augmented visual images are then constructed that include the real-time visual image, the image features and surgical phase information and the augmented image is displayed on the display device.

In a second embodiment, a method for performing surgical procedures using image-guided tools is disclosed. The method comprising receiving in real-time visual images from an imaging system of a surgical field and extracting regions of interest in the surgical field using information provided by an artificial intelligence (AI) model. The method further including selecting a region of interest computed by the AI model and developing by the AI model, selected image features based on surgical instrument used in the region of interest and to classify the phase of the surgical procedure being performed. The method also includes constructing augmented visual images that include the real-time visual images, image features and surgical phase information and displaying the augmented visual images on a display device.

In a third embodiment, a non-transitory computer readable medium is disclosed containing instructions that, when executed by at least one processing device, cause the at least one processing device to receive in real-time visual images from an imaging system of a surgical field during surgery and extract regions of interest in the surgical field using information provided by an artificial intelligence (AI) model. The instructions further executed to select a region of interest computed by the AI model and to develop by the AI model selected image features based on surgical instruments used and to classify the phase of the surgery being performed. The instructions further executed to construct augmented visual images that include the real-time visual images, image features and surgical phase information and display the augmented image on a display device.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which:

FIG. 1 illustrates a block diagram of an example implementation of an image-guided surgical system (IGSS) according to this disclosure;

FIG. 2 illustrates an example schematic for a region-based convolutional neural network (R-CNN) according to this disclosure;

FIG. 3 illustrates a feedback loop formed by the components of the IGSS according to this disclosure;

FIG. 4 illustrates a method for developing and displaying image-guided tools for cataract surgical procedures according to this disclosure;

FIG. 5 illustrates an example display of an augmented image displayed during the capsulorhexis phase of a cataract surgical procedure according to this disclosure;

FIG. 6 illustrates an example display of an augmented image displayed during the phacoemulsification phase of a cataract surgical procedure according to this disclosure;

FIG. 7 illustrates an example display of an augmented image displayed during the cortex removal phase of a cataract surgical procedure according to this disclosure; and

FIG. 8 illustrates an example display of an augmented image displayed when no surgical instrument is inserted into the pupil during a cataract surgical procedure according to this disclosure.

### DETAILED DESCRIPTION

The figures, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

As used herein the term “real-time” refers to the acquisition, processing, and output of images and data that can be used to inform surgical tactics and/or modulate instruments and/or surgical devices during a surgical procedure.

During surgical procedures, unintended interactions between surgical instruments and tissue within the surgical field may have: unintended consequences, some of which may be permanent. Ophthalmic microsurgery, for example, entails the use of mechanical and motorized instruments to manipulate delicate intraocular tissues. Great care must be afforded to tissue-instrument interactions, as damage to delicate intraocular structures such as the neurosensory retina, optic nerve, lens capsule, and corneal endothelium can result in significant visual morbidity. The surgical guidance system described herein provides a feedback loop whereby the location of a surgical instrument in relation to delicate tissues (e.g., ocular tissues) and the effect of instrument-tissue interactions can be used to guide surgical maneuvers.

The present disclosure teaches embodiments capable of autonomously identifying the various steps and phases of phacoemulsification cataract surgery in real-time. The present disclosure uses an artificial intelligence (AI) model using a deep-learning neural network, such as for example, a region-based convolutional neural network (R-CNN) Or a segmentation network (SN) to augment visual images from an operating room imaging system, such as for example, a surgical microscope (SM) or an optical coherence tomography (OCT) imaging system. The AI model intraoperatively identifies the location and size of the pupil for tracking and segmentation, the surgical instruments used in the procedure that have penetrated the pupil and the surgical phase being performed. The AI model provides augmented visual images to the surgeon in real-time identifying the surgical instruments' location in the intraocular compartment, the phase of the surgical procedure and other information and features that aid the surgeon during the surgical procedure. Alternately, the system can also overlay surgical instrument's location, surgical phase, and informational features over visual images input from the imaging system in real-time.

The augmented images or alternatively the overlayed images are outputted to the surgeon to a display device for the surgeon's information and use, for example, to the oculars of an SM, to a display monitor, or an augmented reality headset.

The present disclosure uses an image-guided surgical system (IGSS) to facilitate the delivery of real-time actionable image data and feedback to the surgeon during a surgical procedure. More specifically, the IGSS generates and delivers the computer-augmented images and/or other feedback to the surgeon allowing the surgeon to reliably recognize the location of surgical instruments and tissue boundaries in the surgical field and understand the relationship between the tissue boundaries and the surgical instrument. $

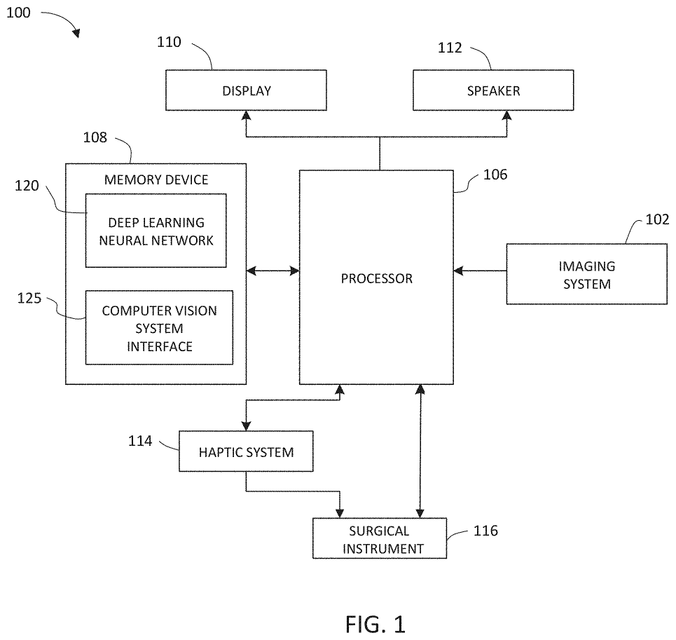

FIG. 1 is a block diagram illustrating, an example IGSS used in the implementation of the disclosed embodiment. The example IGSS 100 includes some elements that are optional, as described below, but reflects the general configurations of such systems. Generally, the IGSS 100 includes an imaging system 102, one or more feedback devices 110,112 and 114, a computer processor 106, and a memory device 108. The feedback devices may include, in various embodiments, a display device 110, an audio speaker 112 or other noise-generating device, such as for example, a piezoelectric buzzer and/or a haptic system 114. It is contemplated that an IGSS 100 would include the display device 110, though the display device 110 may, in various embodiments, provide more or less feedback to the surgeon, and may provide that feedback in a variety of forms, as described below. It is contemplated, for example, that all embodiments of the IGSS 100 may at least show on the display device 110 an image of the surgical field, and that the image is augmented in some fashion to depict, intermittently or continuously, in real-time, within the surgical field, surgical instruments placement, selected features for the surgical phase, surgical templates indicating a recommended tool path or incision location or course that guides the surgeon to make and classification of the surgical phase being performed. The IGSS 100 display device 110 may also display quantitative or qualitative information to the surgeon such as for example, movement or acceleration of surgical instruments and tissues, fluidic parameters such as turbulence, warnings regarding potentially unsafe conditions, such as deviation of a surgical instrument out of the field of view of the surgeon or imaging system, or conditions of turbulent flow associated with surgical complications.

Even though the IGSS 100 is described using separate component elements in this disclosure, it will be well understood by those skilled in the art that in other embodiments certain elements of the IGSS 100 may be omitted and/or combined to provide the same functionality described herein. For example, the imaging system 102 may be integrated with the computer processor 106 and the display device 110. Alternately, the display device 110 may be a part of a stand-alone imaging system, not an integral part of the IGSS 100, that may be interfaced and used with an existing imaging system 102. The display device 110 may also include one or more display devices 110, audio speakers 112, haptic 114 or other feedback device for receiving augmented images and other feedback developed by the IGSS 100.

As was explained earlier, the present disclosure uses an AI model that utilizes a deep-learning neural network, such as for example, a region-based convolutional neural network (R-CNN), a convolutional neural network (CNN) or a segmentation network (SN) to augment visual images from the imaging system 102. The AI model resides in the deep learning neural network (NN) module 120 stored in memory device 108. The memory device 108 also stores a processor operating system that is executed by the processor 106, as well as a computer vison system interface 125 for constructing augmented images. When executing the stored processor operating system, the processor 106 is arranged to obtain the visual images of the surgical field from imaging system 102 and output augment image data, using data provided by the NN 120. The augmented image data is converted to augmented images by the computer vision interface 125 and fed back to the surgeon on the display device 110. The processor 106, may also provide other forms of feedback to the surgeon such as audio alerts or warnings to the speaker 112, or vibrations or rumbles generated by the haptic system 114 to a surface of the imaging system 102 or to the surgical instrument 116. The audio warnings and vibrations alerting the surgeon of the surgical instrument 116 associated with a potential for suboptimal execution or complications such as for example, unintended deviation into a particular location or plane during the surgical procedure.

In one exemplary embodiment the NN module 120 may provide object detection using a selected search for regions based on the following three processes:
1. Find regions in the image that might contain an object. These regions are called region proposals.
2. Extract convoluted neural network features from the region proposals.
3. Classify the objects using the extracted features.

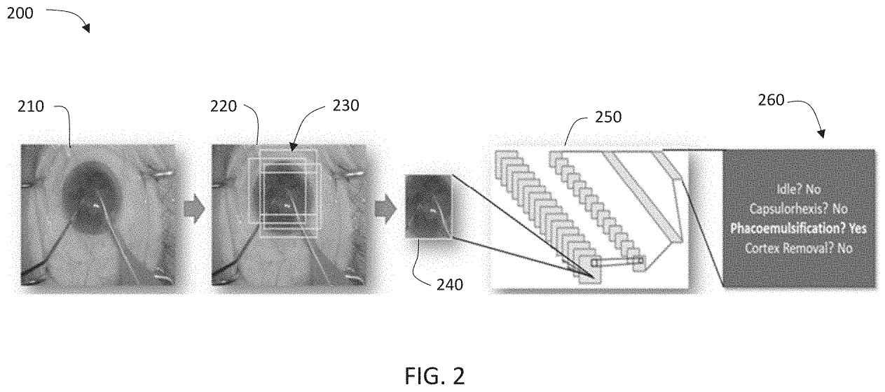

FIG. 2 illustrates a region-based convolutional neural network (R CNN) algorithm 200 that can be used to develop augmented visual images. First an input image 210 is input to the R-CNN algorithm 200 from the imaging system 102. The R-CNN algorithm then generates region proposals 220 using an edge box algorithm 230. The R-CNN algorithm can produce at least 2000 region proposals. The individual region proposals 240 are fed into a convolutional neural network (CNN) 250 that acts as a feature extractor where the output dense layer consists of the features extracted from the input image 210. The extracted features identify the presence of the object within the selected region proposal generating the output 260 of the surgical phase being performed.

In another exemplary embodiment the NN module 120 may provide object detection using a semantic segmentation network. Semantic segmentation can be defined as the process of linking each pixel in a particular image to a class label. The class labels, for example within a surgical field, can be anatomical structures and tissue boundaries and instruments. Through deep learning, the AI model can identify data sets of label images sampled from a training set of ophthalmic surgical procedures. Additionally, semantic segmentation of instruments enables creating an accurate profile of surgical instruments and usage across the surgical procedure. Such class label data sets, along with data for instrument trajectories, can serve as the basis for intraoperative image guidance, as well as image post-processing.

As will be described below, the imaging system 102 may include various forms of image capture technology including for example a two dimensions high-definition SM or a digital stereo microscope (DSM). The DSM is a surgical microscope that relies on at least two cameras that are offset. The two offset images are simultaneously displayed on a display device capable of three-dimensional display which confers stereo viewing to the user. In other embodiments, the IGSS 100 may include other surgical imaging systems such as an intraoperative optical coherence tomography (iOCT) system. In still other embodiments, multiple t pes of imaging systems, such as for example may include both an iOCT system and a SM or DSM system.

The display device 110 may take the form of one or more viewfinders such as the oculars of an SM or DSM, a high-definition display monitor, or a head-mounted display (such as those used for augmented reality and/or virtual reality systems), and the like.

Generally speaking, in embodiments of the IGSS 100 the processor 106 is bi-directionally communicatively coupled to memory device 108, such that the processor 106 may be programmed to execute the software stored in the memory device 108 and, in particular, the visual images input to the IGSS 100 from imaging system 102.

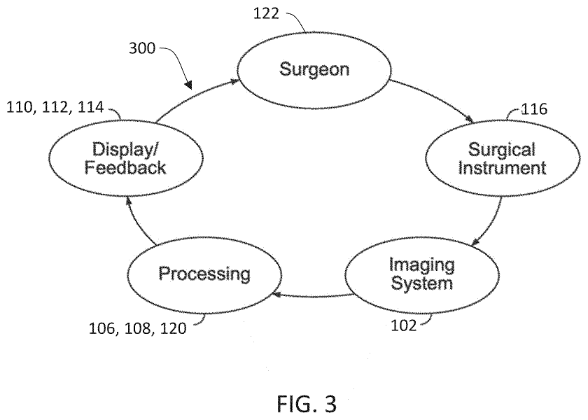

Together the elements of the IGSS 100 shown and described in FIG. 1 form a feedback loop 300, illustrated in block diagram by FIG. 3. In the feedback loop 300, a surgeon 122 provides input in the form of direct manipulation or robotic control to the surgical instrument 116. The surgical instrument 116 appears in the imaging system 102, which provides data to the processing system that comprises the processor 106, the memory device 108 and the NN module 120. The processing system post-process visual images from the imaging system 102 to output augmented images to the display device 110 and haptic feedback to haptic system 114, audio feedback to speakers 112 and/or to control features of certain surgical instruments used during the surgical procedure. The surgeon 122 based on the visual images or other feedback, can modify his or her actions accordingly or the processing system automatically adjust certain features of a surgical instrument 116.

For example, based on the post processed visual images of the surgical instruments and tissue elements in the surgical field, various operational features of the surgical instrumentation 116 can be automatically adjusted, such as for example, adjusting the power to an ultrasonic phaco probe during emulsification of the lens nucleus during cataract surgery. The power driving the ultrasonics can be reduced, modulated, or shut-off in the event that suboptimal or high-risk conditions occur, or if the surgical instrument 116 exhibits unintended deviation into a particular location or plane. Additionally, the fluidics controller used in a cataract surgical system, used with a phaco emulsification probes or irrigation-aspiration probes, and used to aspirate emulsified lens particles, lens material, or intraocular fluids may be automatically modulated by the feedback system to alter the vacuum generated by an associated vacuum pump based on detected changes in the behavior of tissues, surgical instruments, or other parameters of the surgical procedure. For example, the vacuum produced by the pump may be increased when the aspiration instrument is in the center of the surgical field removing hardened emulsified lens particles or decreased as it enters the softer outer lens cortex. In various embodiments of the TOSS 100 illustrated in EEGs 1 and 3, the display device 110 may also include a depiction, in real-time, within the surgical field, of a surgical instrument 116 wielded by the surgeon, and may identify on the display 110 a tip, working end or other salient feature of the surgical instrument 116.

In another embodiment, the feedback from the processing subsystem may also be directly applied (not shown) to the surgical instrument 116 simultaneously as the augmented visual image is displayed to the surgeon 122. This would be useful in situations where the processing system detects a situation wherein eye structures, tissues, or spaces would be violated due to for example, shifting of tissue or patient movement. In such a scenario, haptic feedback such as for example, vibration or rumble could be sent to the surgical instrument 116 held in the surgeon's hand, providing tactile feedback as a warning to the surgeon. In robotic manipulated surgical instruments, the surgical instrument 116 can be automatically retracted from the area of concern by the motorized instrument manipulators or prevented from ingress into a particular risk or prohibited location or plane. Alternately, in a haptic-mediated surgical system, resistance to movement of the surgical instrument could be induced by the haptic system 114 to prevent movement of the surgical instrument 116 into a particular risk or prohibited location or plane.

As described above, the imaging systems 102 are located and used outside of the eye or body to view the surgical field. In some embodiments, particularly those configured to be employed during ocular surgeries such as cataract surgery, the imaging systems may include an SM system, a DSM system an iOCT system, or combination of such imaging systems. The imaging system 102, when configured to be employed during ophthalmic surgeries, may be operative to identify any of a variety of ocular tissues including, lens tissue, cracking defects in the ins fragments, corneal tissue, the corneal limbus, iris tissue, the pupil, the anterior chamber, the lens capsular bag, the capsulorrhexis margins, the hydrodissection light reflex, the fundus red reflex, position and centration of the intraocular lens implant, etc.

In the presently described embodiment, visual images in the form of digital image data from the imaging system 102 is input into the NN module 120 of the AI model. The NN module 120 may analyze the digital image data to determine the tissue boundaries and/or layers, so that the data output by the AI model indicates the tissue boundaries/layers that may be added to the raw image data for display to the surgeon. The displayed tissue boundaries/layers assisting the surgeon in avoiding contact between the surgical instrument 116 and sensitive tissues. Additionally, the AI model may also provide instrument guidance for spatial orientation and/or optimizing instrument parameters related to function such as aspiration and associated vacuum/flow rates, ultrasound parameters, etc.

The AI model automatically segments the image data using a deep learning approach using the algorithm of the NN module 120. The AI model receives as an input the digital image data obtained from the imaging system 102 and provides a segmentation probability map of the location of the tissue in question (e.g., the retina, the lens, etc.). The segmentation probability map may also provide utility measurement such as for example the relative area change and/or volume change of the tissue of the retina or the lens between different images. The utility measurement of the area of change/or volume of change may be used by the surgeon to estimate how much the tissue's area is changing, therefore providing information about the amount of stress the tissue is undergoing at a particular instant in the procedure. The relative change in height of the tissue between images provides a similar, but different, type of stress indication. The position and/or motion of the tissue relative to adjacent ocular tissues, identify occlusion of an instrument by a tissue. Using the algorithms described herein, the segmentation is achieved at a frame rate of up to or in excess of 60 frames-per-second, allowing the presentation to the surgeon of real-time segmented images as augmented visual images.

The NN module 120 algorithms of this embodiment use datasets, with training performed in a supervised or self-supervised manner, which include both the source visual image and the segmented images. The trained AI model implementing the NN module 120 uses digital image data from the imaging system, labelled by experts, as a training set, in the case of supervised learning.

In still another embodiment, the AI model may also be used to pre-process the input visual images received from the imaging system 102. The Pre-processing of the input images improves image resolution for the surgeon for use in real-time, as a form of image enhancement to allow the surgeon to appreciate details of the image that may otherwise be obscured or not apparent in un-processed imaging.

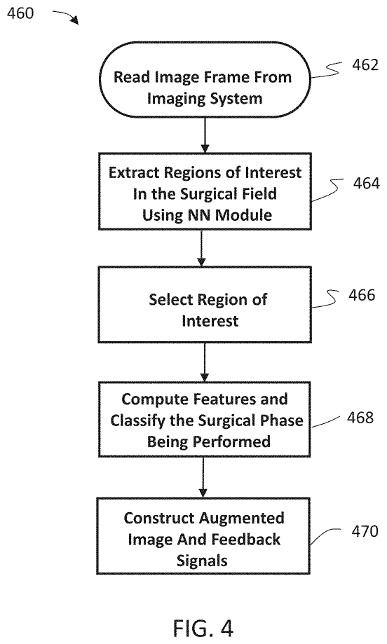

FIG. 4 illustrates a flow chart depicting a method 460 that implements an exemplary embodiment for cataract surgical procedures employing the image-guided tool of the present disclosure. In step 462, the processor 106 receives digital image data representing visual images captured in real-time from the imaging system 102. The digital image data along with the AI trained data from the NN module 120 is input to the processor 106 in step 464. The processor 106 identifies and outputs data identifying region proposals for the pupil's location and area as described earlier in the discussion for R-CNN.

In step 466, the processor 106 using a calculated R-CNN classification for the pupil, selects a region of interest based on an optimal pupil location and area. The processor 106 using the AI trained data from NN module 120 computes in step 468, selected features for the surgical phase and identifies the classification of the phase of the cataract procedure being performed. The features and classification are based on the type of surgical instruments located within the pupil captured by the imaging system 102.

In step 470 augmented visual images are constructed by the computer vision systems interface 125. The augmented visual images are output to the surgeon's SM eyepiece or the display device 110. Additionally, feedback signals may also be output including haptic and/or audible signals applied to the haptic system 114 or speaker 112. The method described by steps 462-470, are made for each image frame captured from the imaging system 102, always acquiring the last available frame from the imaging system 102 at a minimum video streaming rate of 60 frames per second, or other frame rates that mat be suitable.

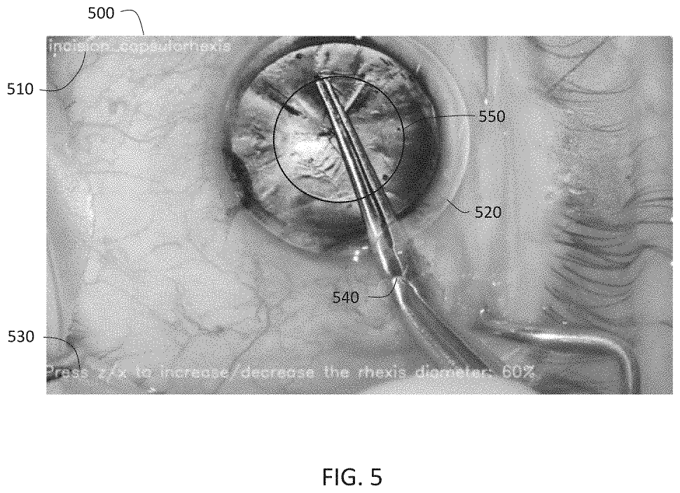

The feedback returned by the exemplary imaging system can include guidance for the optimal creation of a capsulorrhexis, including size, location, centration, and symmetry of the rhexis as is seen in FIG. 5. Capsulorrhexis parameters and guidance recommendations may be altered in real time based upon real-time image-based feedback on the evolving capsulorrhexis as executed during the surgery. The augmented visual image 500 provides an identification of the phase of the surgical procedure 510, based on the type of surgical instrument 540 used in the procedure. Image 500 displays a rhexis template 550 and guidance instructions 530 to guide and instruct the surgeon for adjustment of the rhexis diameter as features for this surgical phase. Further features include visual feedback of the pupil boundary 520 where local contrast enhancement may be applied. FIG. 5 illustrates the surgical instrument 540 penetrating the outer tissue of the eye, either the sclera or the cornea.

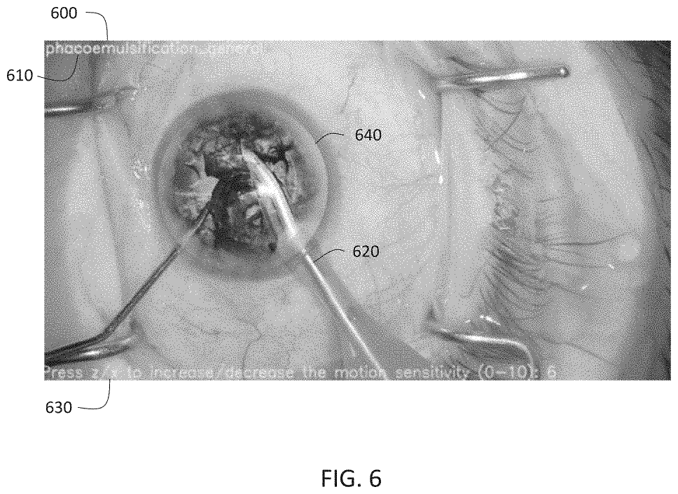

FIG. 6 displays the augmented visual image 600 for surgical guidance during disassembly and removal of the lens nucleus via phacoemulsification. Identification of the procedure is identified at 610 based on the surgical instrument 620 used in the procedure in this surgical phase, excessive eye movement warning, computation of amount of remaining lens fragments via tissue segmentation, and estimation of turbulent flow conditions via tracking of the motion of lens fragments may be visually indicated to the surgeon by displaying a visual cue 640 where local contrast enhancement may be applied. Visual cue 640 indicates when turbulence or any brusque movement of the surgical instrument 620 is identified. The feedback thresholds for the guidance parameters 630 may be modulated by the surgeon and are provided as a visual feature, during the relevant surgical phase.

The tracking of turbulent flow during this surgical phase uses visual feedback from the NN module 120 or from computer vision techniques that estimate turbulent flow, the movement and tracking of the surgical instruments and lens fragments. Biomarkers associated with surgical risk may also be detected as features and information provided to the surgeon in this surgical phase. For example, rapid changes in pupillary size, reverse pupillary block, trampoline movements of the iris, spider sign of the lens capsule, and a change in the fundus red reflex may be identified and provided as feedback in real time to the surgeon. In addition, instrument positioning associated with surgical risk, such as decentration of the tip of the phacoemulsification needle outside of the central zone, duction movements of the globe away from primary gaze during surgery and patient movement relative to the surgical instruments may be identified and provided as feedback to the surgeon as either visual warning images, or haptic and audio alarms in real-time.

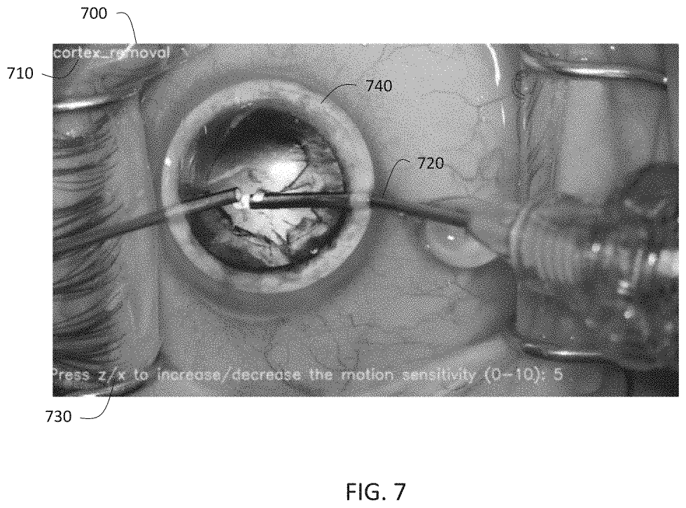

FIG. 7 displays the augmented image 700 for cortex removal. Based on the instrument 720 used, feedback information is presented to the surgeon including the procedure phase 710. Instrument 720 movement warnings and motion sensitivity thresholding 730 is also provided to aid in the removal of cortical fibers. The augmented image 700 can have contrast equalization applied in the form of a local image enhancement 740 where local contrast enhancement is applied. As is seen in FIG. 7, a visual cue 740 is applied within and around the area of the pupil.

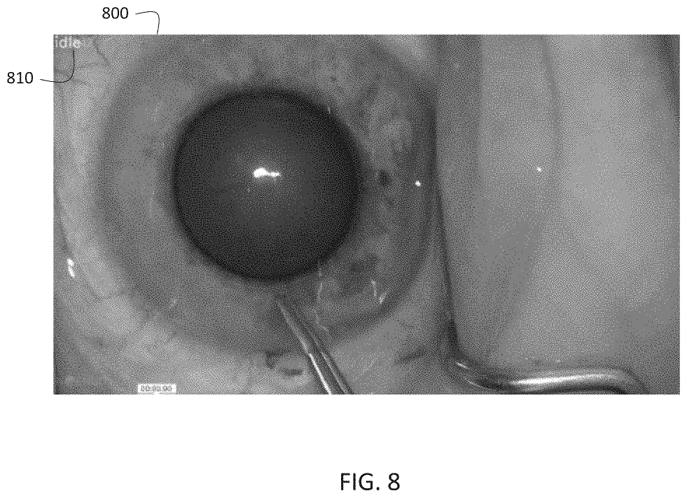

When no instrument is inserted into the pupil, the CNN recognizes the image 800 phase being performed as “idle” as shown at 810 of FIG. 8.

In some embodiments, various functions described in this patent document are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

It may be advantageous to set forth definitions of certain words and phrases used throughout this patent document. The terms “application” and “program” refer to one or more computer programs, software components, sets of instructions, procedures, functions, objects, classes, instances, related data, or a portion thereof adapted for implementation in a suitable computer code (including source code, object code, or executable code). The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in the present application should not be read as implying that any particular element, step, or function is an essential or critical element that must be included in the claim scope. The scope of patented subject matter is defined only by the allowed claims. Moreover, none of the claims is intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” or “controller” within a claim is understood and intended to refer to structures known to those skilled in the relevant art, as further modified or enhanced by the features of the claims themselves, and is not intended to invoke 35 U.S.C. § 112(f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An image-guided tool for surgical procedures comprising:
a processor;
a display device coupled to the processor;
an imaging system coupled to the processor;
a memory device, coupled to the processor storing instructions executable by the processor, the memory device including an artificial intelligence (AI) model, to cause the processor to:
receive, from the imaging system, visual images in real-time of a surgical field during the surgical procedure;
extract regions of interest in the surgical field using information provided by the AI model;
select a region of interest computed by the AI model;
compute image features for the surgical phase being performed;
construct augmented visual images; and
display the augmented visual images on the display device.

2. The image-guided tool of claim 1, wherein the imaging system is located external to the surgical field and coupled to the processor.

3. The image-guided tool of claim 2, wherein the AI model computes image features based on a surgical instrument used in the surgical procedure.

4. The image-guided tool of claim 3, wherein the AI model classifies the phase of the surgical procedure being performed based on the visual images of the surgical instrument used in the surgical field.

5. The image-guided tool of claim 4, wherein the augmented visual images include the real-time visual images of the surgical field during the surgical procedure, the image features and the phase of the surgical procedure computed by the AI model.

6. The image-guided tool of claim 4, wherein the AI model provides feedback signals to an auditory device the auditory device providing an audio warning when the surgical instrument approaches deviates into a particular location or plane during the surgical procedure.

7. The image-guided tool of claim 4, wherein the AI model provides haptic feedback signals to a haptic device, the haptic device vibrating the surgical instrument when the surgical instrument approaches or deviates into a particular location or plane during the surgical procedure.

8. The image-guided tool of claim 4, wherein the surgical instrument is robotically manipulated and the AI model provides feedback signals to the robotic surgical instrument, wherein the robotic surgical instrument is automatically retracted from the surgical field when the robotic surgical instrument approaches or deviates into a particular location or plane during the surgical procedure.

9. The image-guided tool of claim 4, wherein operational features of the surgical instrument are adjusted by feedback signals from the AI model.

10. The image guided tool of claim 2, wherein the AI model is a region-based convolutional neural network (R-CNN).

11. The image guided tool of claim 2, wherein the AI model is a segmentation network (SN).

12. The image guided-tool of claim 2, wherein the display device is a surgical microscope.

13. The image guided-tool of claim 2 wherein the display device is a display monitor or an augmented reality headset.

14. A method for performing surgical procedures using an image-guided tool, the method comprising:
receiving in real-time visual images from an imaging system of a surgical field;
extracting regions of interest in the surgical field using information provided by an artificial intelligence (AI) model;
selecting a region of interest computed by the AI model;
developing by the AI model selected image features based on the surgical instrument used in the region of interest and classifying the phase of the surgical procedure being performed;
constructing augmented visual images that includes the real-time visual images, image features and surgical phase information; and
displaying the augmented visual images on a display device.

15. The method of claim 14, wherein the AI model provides feedback signals to an auditory device the method further comprising:
producing an audio warning by the auditory device when the surgical instrument approaches or deviates into a particular location or plane during the surgical procedure.

16. The method of claim 14, wherein the AI model provides haptic feedback signals to a haptic device the method further comprising:
vibrating the surgical instrument when the surgical instrument approaches or deviates into a particular location or plane during the surgical procedure.

17. The method of claim 14, wherein the surgical instrument is robotically manipulated and the AI model provides a feedback signal to the robotic surgical instrument, the method further comprising:
automatically retracting the robotic surgical tool from the surgical field when the robotic surgical tool approaches or deviates into a particular location or plane during the surgical procedure.

18. The method of claim 14, wherein the AI model is a region-based convolutional neural network (R-CNN), the R-CNN operates to:
find regions in the visual images that may contain an object and provide region proposals;
extract convoluted neural network features from the region proposals;
classify the objects using the extracted features; and
construct augmented visual images of the surgical field using the objects classified from the extracted features.

19. The method of claim 14, wherein the AI model is a segmentation network (SN), the SN operates to:
identify data sets of label images sampled from a training set of ophthalmic surgical procedures;
creating an accurate profile of the surgical instruments and their usage for the ophthalmic surgical procedure;
develop class labels within the ocular surgical field for the anatomical structures, tissue boundaries and tools;
identify through deep learning a training set of ophthalmic surgical procedures; and
construct augmented visual images of the surgical field using the objects classified from the data sets of label images.

20. A non-transitory computer readable medium containing instructions that when executed by at least one processing device, cause the at least one processing device to:
receive in real-time visual images from an imaging system of a surgical field;
extract regions of interest in the surgical field using information provided by an artificial intelligence (AI) model;
select a region of interest computed by the AI model;
develop by the AI model selected image features based on the surgical instruments used in the region of interest and classify the phase of the surgery being performed;
construct augmented visual images that include the real-time visual images, image features and surgical phase information; and
display the augmented visual images on a display device.
